SEQUENCE LISTING (21 sequences)

SEQ ID NO: 1 - protein, 15 aa, Artificial Sequence
  Synthetic Peptide
MVRIKPASANKPSDD

SEQ ID NO: 2 - protein, 15 aa, Artificial Sequence
  Synthetic Peptide
MVRIKPGTANKPSDD

SEQ ID NO: 3 - protein, 15 aa, Artificial Sequence
  Synthetic Peptide
MVRIKPGTANKPTDD

SEQ ID NO: 4 - protein, 15 aa, Artificial Sequence
  Synthetic Peptide
MVRIKPGSGNKPSDD

SEQ ID NO: 5 - protein, 15 aa, Artificial Sequence
  Synthetic Peptide
MVRIKPATANKPSDD

SEQ ID NO: 6 - protein, 15 aa, Artificial Sequence
  Synthetic Peptide
MVRIKPGTGNKPSDD

SEQ ID NO: 7 - protein, 15 aa, Artificial Sequence
  Synthetic Peptide
MVRIKPGTGNKPTDD

SEQ ID NO: 8 - protein, 15 aa, Artificial Sequence
  Synthetic Peptide
MVRIKPATANKPTDD

SEQ ID NO: 9 - protein, 15 aa, Artificial Sequence
  Synthetic Peptide
MVRIKPATGNKPTDD

SEQ ID NO: 10 - protein, 15 aa, Artificial Sequence
  Synthetic Peptide
MVRIKPATGNKPSDD

SEQ ID NO: 11 - protein, 14 aa, Artificial Sequence
  Synthetic Peptide
VRLKPASANKPSED

SEQ ID NO: 12 - protein, 14 aa, Artificial Sequence
  Synthetic Peptide
VRLKPASGNKPSED

SEQ ID NO: 13 - protein, 14 aa, Artificial Sequence
  Synthetic Peptide
VRLKPGSGNKPSED

SEQ ID NO: 14 - protein, 13 aa, Artificial Sequence
  Synthetic Peptide
VRLKPATGNKPTE

SEQ ID NO: 15 - protein, 14 aa, Artificial Sequence
  Synthetic Peptide
VRLKPGTANKPTED

SEQ ID NO: 16 - protein, 14 aa, Artificial Sequence
  Synthetic Peptide
VRLKPATANKPTED

SEQ ID NO: 17 - protein, 14 aa, Artificial Sequence
  Synthetic Peptide
VRLKPGTGNKPTED

SEQ ID NO: 18 - protein, 14 aa, Artificial Sequence
  Synthetic Peptide
VRLKPGTANKPSED

SEQ ID NO: 19 - protein, 14 aa, Artificial Sequence
  Synthetic Peptide
VRIKPGTANKPSED

SEQ ID NO: 20 - protein, 15 aa, Artificial Sequence
  Synthetic Peptide
  VARIANT (1)..(1): X can be M or no amino acid
  VARIANT (4)..(4): X can be I or L
  VARIANT (7)..(7): X can be G or A
  VARIANT (8)..(8): X can be S or T
  VARIANT (9)..(9): X can be A or G
  VARIANT (13)..(13): X can be S or T
  VARIANT (14)..(14): X can be D or E
XVRXKPXXXNKPXXD

SEQ ID NO: 21 - protein, 15 aa, Artificial Sequence
  Synthetic peptide
MVRIKPGSANKPSDD

(19) United States
(12) Patent Application Publication
(10) Pub. No.: US 2024/0293501 A1
(43) Pub. Date: Sep. 5, 2024

(54) Reagents, Pharmaceutical Formulations, and Methods for Treating and Preventing Viral Infection

(71) Applicants: Michael J. PAIDAS, Miami, FL (US); Arumugam JAYAKUMAR, Miami, FL (US); University of Miami, Miami, FL (US)

(72) Inventors: Michael J. Paidas, Miami, FL (US); Arumugam Jayakumar, Miami, FL (US)

(21) Appl. No.: 18/002,480

(22) PCT Filed: Jun. 21, 2021
(86) PCT No.: PCT/US2021/038234

§ 371 Date: Dec. 19, 2022

Related U.S. Application Data
(60) Provisional application No. 63/041,100, filed on Jun. 18, 2020.

(51) Int. Cl. A61K 38/10; A61P 31/12

(52) U.S. Cl. CPC A61K 38/10; A61P 31/12

(57) ABSTRACT

This invention provides reagents, pharmaceutical formulations comprising such reagents, and methods for preventing and treating viral infections, specifically coronavirus infections, and in particular infections in humans with COVID-19.

### REFERENCE STATEMENT

This application is a U.S. national phase application under 35 U.S.C. 371 of International Patent Application No. PCT/US2021/038234, filed Jun. 21, 2021, which claims priority to U.S. Provisional Patent Application No. 63/041,100 filed on Jun. 18, 2020, entitled “REAGENTS, PHARMACEUTICAL FORMULATIONS AND METHODS FOR TREATING AND PREVENTING VIRAL INFECTION,” the disclosure of each of which is incorporated herein in its entirety.

### STATEMENT REGARDING SEQUENCE LISTING

A computer readable form of the Sequence Listing is filed with this application by electronic submission and is incorporated into this application by reference in its entirety. The sequence listing submitted herewith is contained in the text file created Oct. 10, 2023, entitled “20-989-WO-US_Sequence-Listing_ST25.txt” and 5,767 bytes in size.

### FIELD OF THE INVENTION

This disclosure relates generally to reagents, pharmaceutical formulations comprising such reagents, methods for using said reagents to make said pharmaceutical formulations, and methods for using said pharmaceutical formulations for preventing and treating viral infections, specifically coronavirus infections, and in particular infections in humans with SARSCOV-2, i.e., COVID-19. The invention discloses a particular synthetic peptide disclosed by its component amino acid sequence. The invention also provides pharmaceutical formulations of the peptide, alone or in combination with suitable excipients and other pharmaceutical formulating agents. Methods for using such pharmaceutical formulations are provided for treating and preventing viral disease.

### BACKGROUND

Coronavirus 2019 Disease (COVID-19) was first identified in Wuhan Provence, China in December 2019. In March 2020, the World Health Organization (WHO) designated the novel coronavirus outbreak a pandemic. As of Jun. 16, 2020, there have been 7,941,791 confirmed cases of COVID-19, including 434,796 deaths, reported to WHO. COVID-19 is caused by the virus SARS-COV-2 that spreads from person-to-person via close contact, respiratory droplets and contaminated surfaces or objects from both symptomatic and asymptomatic individuals. The virus enters host cells by binding a cell-surface host cell receptor, angiotensin-converting enzyme 2 (ACE2). No effective treatment is available. Currently, there are no specific antiviral strategies to combat the viral infection, although treatment strategies are currently under investigation and some vaccines have been developed.

SARS-COV-2 is a β-coronavirus, enveloped non-segmented positive-sense RNA virus. The virus genome encodes 4 essential structural proteins, spike (S) glycoprotein (SGP), small envelope (E), matrix (M), nucleocapsid (N), and several accessory proteins; a diagram of the structure is shown in FIG. 8.

SARS-COV-2 uses the same receptor (angiotensin-converting enzyme 2, ACE2) as that for SARS-COV-1. The viral SGP binds to ACE2, allowing viral entry into the cell. Preventing SGP binding to ACE2 can reduce SARS-COV-2 transmission. Thus, there remains a need in the art for reagents and methods that prevent or treat coronavirus infection, in particular COVID-19, by inhibiting or preventing binding of SGP to ACE2.

### SUMMARY

This disclosure provides reagents, pharmaceutical formulations, and methods for treating or preventing coronavirus infection in humans, including SARS-COV-2 infection. The disclosure also provides reagents, pharmaceutical formulations, and methods for treating or preventing disease characterized by inflammation, multi-organ failure, oxidative stress, infection, lipopolysaccharide effects, edema, water channels, and/or coagulation or hemorrhage or thrombosis in humans. In a first aspect, the invention provides a pharmaceutical formulation for treating or preventing a coronavirus infection in an animal or human comprising a therapeutically effective amount of a peptide, wherein the formulation comprises peptides having an amino acid sequence comprising: (M)nVR(I/L)KP(G/A)(S/T)(A/G)NKP(S/T)(D/E)D (SEQ ID NO: 20), wherein n=0 or 1, wherein the peptide does not have the amino acid sequence MVRIKPGSANKPSDD (SEQ ID NO: 21) and wherein variant amino acid sequence residues are set forth in the alternative at each substituted position. In a second aspect is a peptide having an amino acid sequence that is MVRIKPASANKPSDD (SEQ ID NO: 1); MVRIKPGTANKPSDD (SEQ ID NO: 2); MVRIKPGTANKPTDD (SEQ ID NO: 3); MVRIKPGSGNKPSDD (SEQ ID NO: 4); MVRIKPATANKPSDD (SEQ ID NO: 5); MVRIKPGTGNKPSDD (SEQ ID NO: 6); MVRIKPGTGNKPTDD (SEQ ID NO: 7); MVRIKPATANKPTDD (SEQ ID NO: 8); MVRIKPATGNKPTDD (SEQ ID NO: 9); MVRIKPATGNKPSDD (SEQ ID NO: 10); VRLKPASANKPSED (SEQ ID NO: 11); VRLKPASGNKPSED (SEQ ID NO: 12); VRLKPGSGNKPSED (SEQ ID NO: 13); VRLKPATGNKPTED (SEQ ID NO: 14); VRLKPGTANKPTED (SEQ ID NO: 15); VRLKPATANKPTED (SEQ ID NO: 16). VRLKPGTGNKPTED (SEQ ID NO: 17); VRLKPGTANKPSED (SEQ ID NO: 18); or VRIKPGTANKPSED (SEQ ID NO: 19) or salts or derivatives thereof.

In particular embodiments, the peptide has the amino acid sequence MVRIKPASANKPSDD (SEQ ID NO: 1) (termed SPIKENET herein) and salts and derivatives thereof.

In another aspect is a pharmaceutical formulation for treating or preventing a coronavirus infection in an animal or human comprising a therapeutically effective amount of a peptide and one or a plurality of excipients, adjuvants, or other formulation components.

Further disclosed is a method of treating or preventing infection of a human or animal with a coronavirus, the method comprising administering to a human in need thereof a pharmaceutical formulation that includes the peptides noted herein.

In further particular embodiments, the pharmaceutical formulation can be administered intranasally, intravenously, mucosally, orally, subcutaneously or intramuscularly. Said pharmaceutical formulations are provided wherein the formulation can be administered two or more times per day and in specific embodiments wherein the pharmaceutical formulation is administered to a human. Most particularly the pharmaceutical formulations and methods provided herein are for prevention and/or treatment of infection to SARS-COV-2.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1 illustrates a stick diagram of the predicted structure of the SPIKENET peptide with numbering scheme.

FIG. 2 is a representation produced using computational protein-peptide docking approach shows highly specific binding affinity of the SPIKENET peptide to the receptor binding domain (RBD) (i.e., ACE2 receptor binding domain) of the SARS-COV-2 spike glycoprotein (SGP). RBD is showed in surface diagram with charged potential. SPIKENET peptide (shown as a stick diagram) specifically binds with the ACE2 binding domain of SGP.

FIG. 3 illustrates a surface diagram of SPIKENET peptide (green color) at the RBD indicate the occupied area of the peptide on the RBD, which specifies that the SPIKENET has high affinity binding on SGP.

FIG. 4 illustrates the SPIKENET peptide binding to the ACE2 binding domain of viral SGP, wherein the SPIKENET peptide is shown in spheres diagram.

FIG. 5 shows the receptor binding domain of SGP (in line diagram), and SPIKENET peptide in spheres diagram.

FIG. 6 shows the SPIKENET peptide in a ball-and-stick diagram along with charged potential surface diagram of receptor binding domain of SGP.

FIG. 7 is a “Dimplot” diagram of RBD (below the line) and SPIKENET peptide (above the line) that describes the non-bonded interactions between the binding site residues of the RBD and SPIKENET peptide. Dotted green lines represent the hydrogen bonds between the residues. Red color half spheres symbolize the hydrophobic interactions between the binding site residues.

FIG. 8 is an illustration of the SARS-COV-2 virus (COVID-19) with the line structure of Angiotensin Converting Enzyme 2-Receptor, the virus spike (S) protein, and the predicted receptor binding region between ACE-2 receptor and the viral protein.

FIG. 9 demonstrates the effect of SPIKENET (SPK) on LPS-induced IL-6 release in primary cultures of rat brain microglia. Exposure of primary microglia to LPS (24 h) showed an increase in IL-6 level in cell culture medium, and such increase was diminished by treatment of cells (30 min. post-treatment) with SPIKENET. *p<0.005 versus control; †p<0.05 versus LPS. C, control; LPS, lipopolysaccharide.

FIG. 10 demonstrates the effect of SPIKENET (SPK) on LPS-induced free radical generation in primary cultures of rat brain microglia. Exposure of primary microglia to LPS (24 h) showed an increase in DCF fluorescence, and such increase was diminished and blocked by treatment of cells (30 min post-treatment) with SPIKENET. *p<0.05 versus control; †p<0.05 versus LPS. C, control; AIU, arbitrary intensity units; LPS, lipopolysaccharide.

FIG. 11 demonstrates the effect of SPIKENET (SPK) on hydrogen peroxide (H₂O₂)-induced oxidative stress (protein carbonyl formation) (12 hr) (A) and LPS-induced LDH release (36 hr) (B) in primary cultures of rat brain microglia, as well as H₂O₂ induced increase in cell volume (24 hr) in primary cultures of rat brain astrocytes (C). SPK significantly diminished these effects in glial cells (30 min post-treatment). *p<0.05 versus control; †p<0.05 versus LPS. C, control; LPS, lipopolysaccharide

FIG. 12 demonstrates the protein docketing approach showing highly specific SPIKENET binding affinity to the CEACAM1a (CCM) binding domain of the MHV-1 spike protein (MHV-1-S1).

FIG. 13 demonstrates SPK binding affinity to the (CCM) binding domain of the MHV-1-S1, but not with the CCM.

FIG. 14 demonstrates MHV-1 inoculated mice display signs of sickness. FIG. 14A) About 40% mice showed signs of sickness on day 2 post-MHV-1 inoculation, and the number of mice exhibiting signs of symptoms increases gradually (up to 75%). The remaining 25% of mice did not show any symptoms of sickness (observed for up to 150 days). (FIG. 14B) MHV-1 inoculated mice showed symptoms of sickness (from stages I-VI) from days 2-12 and beyond. Mild to moderate symptoms (stages I-III) were observed from days 2-4. The MHV-1 infected mice showed severe sickness (stages IV-VI) from 6 days onward. Further, 60% of animals died from days 7-12 (n=16). Symptoms were scored by stages: 0) no symptoms, I) drowsy and lack of movement, II) slightly ruffled fur and altered hind limb posture, III) ruffled fur and mildly labored breathing, IV) ruffled fur, inactive, moderately labored breathing, V) ruffled fur, obviously labored breathing and lethargy, and VI) moribund and death.

FIG. 15 demonstrates MHV-1 inoculated mice displayed disease severity. FIGS. 15A & 15B. Normal mice. FIGS. 15C, 15D & 15G. Representative Mouse from MHV-1 inoculated group showed venous thrombosis consistent with symptoms of patients associated with COVID-19 (FIGS. 15E and 15F) and FIG. 15C, mouse inoculated with MHV-1 exhibit insufficient respiration. Treatment of MHV-1 mice with 5 mg/kg SPIKENET reversed the symptoms (FIGS. 15H & 15I).

FIG. 16 demonstrates MHV-1 infected mice losses body weight: MHV-1 inoculated mice had lost their body weight from 20-40% from days 3-8 that correspond well with the severity of the disease condition (n=16).

FIG. 17 demonstrates organ histology in infected and uninfected mice and the effect of SPIKENET: Representative histological images of haematoxylin and eosin (H&E) stained lung tissue sections of normal mouse (FIG. 17A) and infected mouse lung (FIG. 17B). MHV-1-infected mice lung shows arterial endothelial swelling (hypertrophy, long arrow), inflammation/granular degeneration of cells (short arrow) and migration of leukocytes (arrowhead) into lung. Peribronchiolar interstitial infiltration, bronchiole epithelial cell necrosis and necrotic cell debris within alveolar lumens, alveolar exudation, infiltration, hyaline membrane formation and alveolar hemorrhage with red blood cells within the alveolar space and interstitial edema were observed in MHV-1-infected mice. Liver, kidney, heart, and brain tissue degenerative changes were also observed in MHV-1-infected mice. Representative images of H&E stained tissue sections are presented. MHV-1-infected mice at day 6 showed liver hepatocytes degeneration, severe cells necrosis (FIG. 17E, long arrows) and hemorrhagic changes (short arrows) when compared to uninfected mice (FIG. 17D); Uninfected kidney (FIG. 17G) and tubular epithelial cells degenerative changes and vacuolation, and peritubular vessels congestion (FIG. 17H & 17I); Severe interstitial edema (arrows), vascular congestion and dilation (arrow heads) and red blood cells infiltrating between degenerative myocardial fibers were seen in the MHV-1 infected mice heart (FIG. 17L & FIG. 17M), as compared to uninfected mice (FIG. 17K). Vacuolation of cerebral matrix (arrows, FIG. 17Q) and cytotoxic edema (arrow heads, FIG. 17Q), congested blood vessels with perivascular edema (long arrows, FIG. 17P), as well as cytotoxic edema (short arrows, FIG. 17P) were seen in MHV-1 infected mice brain cortex. Similar changes were also observed in hippocampus (arrows, FIG. 17T & FIG. 17U). FIG. 17O & FIG. 17S, uninfected mice. These changes were absent or reduced in SPIKENET-treated mice (5 mg/kg by wt.) (FIG. 17C, FIG. 17F, FIG. 17J, FIG. 17N, FIG. 17R and FIG. 17V).

FIG. 18 demonstrates oxidative stress post-MHV-1 infection in mice. Representative immunofluorescence images from 4 individual animals show an increase in levels of 4-hydroxynonenol (4-HNE) and malondialdehyde (MDA) in lung, liver, kidney, brain and heart, and that treatment of MHV-1 inoculated mice with SPK (5 mg/kg) prevented such increase. Quantitation shows only 4-HNE and MDA levels in MHV-1 infection.

FIG. 19 demonstrates organ edema. Wet-to-dry ratio of the lung, liver, kidney, and heart from normal (Control; Cont.) and MHV-1 inoculated mice. Data obtained from 4 individual animals.

FIG. 20 demonstrates aquaporins (AQP1 and 4 level) in multiple organs post-MHV-1 infection in mice. Representative immunofluorescence images from 4 individual animals show an increase in levels of AQP1 and 5 in all organs except that the AQP1 levels are reduced in lung, and that treatment of MHV-1 inoculated mice with SPK (5 mg/kg) normalized such changes. Quantitation shows only AQP levels in MHV-1 infection.

FIG. 21 demonstrates AQP5 and 8 level in multiple organs post-MHV-1 infection in mice. Representative immunofluorescence images from 4 individual animals show an increase in levels of AQP5 and 8 lung, liver, kidney, brain and heart, and that treatment of MHV-1 inoculated mice with SPK (5 mg/kg) prevented such increase. Quantitation shows only AQP levels in MHV-1 infection.

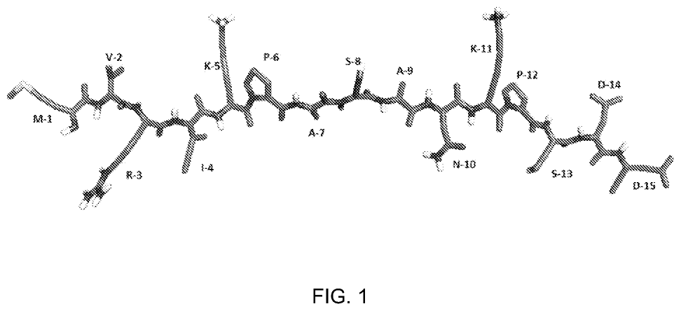

### DETAILED DESCRIPTION OF PREFERRED EMBODIMENTS

This invention provides peptides and pharmaceutical formulations comprising soluble peptides characterized by amino acid sequence, as well as methods for using such formulations for preventing or treating coronavirus infection in humans, particularly COVID-19. In certain embodiments as set forth with further specificity below, the methods provided herein comprise the following generally described steps of the disclosed methods.

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

In addition, any of the compositions or methods provided, disclosed, or described herein can be combined with one or more of any of the other compositions and methods provided, disclosed, or described herein.

### 1. Definitions

Unless defined otherwise, all technical and scientific terms used herein have the meaning commonly understood by a person skilled in the art to which the claimed invention belongs. The terminology used herein is for describing particular embodiments only and is not intended to be limiting of the claimed invention. All technical and scientific terms used herein have the same meaning.

The following references provide those of skill in the art with a general understanding of many of the terms used herein (unless defined otherwise herein): Singleton et al., Dictionary of Microbiology and Molecular Biology, 3rd ed. (Wiley, 2006); Walker, The Cambridge Dictionary of Science and Technology (Cambridge University Press, 1990); Rieger et al., Glossary of Genetics: Classical and Molecular, 5th ed. (Springer Verlag, 1991); and Hale et al., Harper Collins Dictionary of Biology (HarperCollins Publishers, 1991). Generally, the procedures or methods described herein, and the like are common methods used in the art. Such standard techniques can be found in reference manuals such as, for example, Green et al., Molecular Cloning: A Laboratory Manual, 4th ed. (Cold Spring Harbor Laboratory Press, 2012), and Ausubel, Current Protocols in Molecular Biology (John Wiley & Sons Inc., 2004).

The following terms may have meanings ascribed to them below, unless specified otherwise. However, it should be understood that other meanings known or understood by those having ordinary skill in the art are also possible, and within the scope of the claimed invention. All publications, patent applications, patents, and other references mentioned or discussed herein are expressly incorporated by reference in their entireties. In the case of conflict, the present specification, including definitions, will control. If there is a conflict between any of the incorporated references and the instant specification, the specification shall control. In addition, the materials, methods, and examples are illustrative only and not intended to be limiting. Any particular embodiment of the present invention that falls within the prior art can be explicitly excluded from any one or more of the claims. Because such embodiments are deemed to be known to one of ordinary skill in the art, they can be excluded even if the exclusion is not set forth explicitly herein. Any particular embodiment of the invention can be excluded from any claim, for any reason, whether or not related to the existence of prior art.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. As used herein, the term “or” means, and is used interchangeably with, the term “and/or” unless context clearly indicates otherwise.

The invention includes embodiments in which exactly one member of the group is present in, employed in, or otherwise relevant to a given product or process. The invention includes embodiments in which more than one, or all of the group members are present in, employed in, or otherwise relevant to a given product or process.

As used herein, the term “including” means, and is used interchangeably with, the phrase “including but not limited to.”

As used herein, the term “such as” means, and is used interchangeably with, the phrase “such as, for example” or “such as but not limited.”

Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about. Percentages disclosed herein can vary in amount by ±10, 20, or 30% from values disclosed and remain within the scope of the contemplated invention.

Ranges provided herein are understood to be shorthand for all of the values within the range including the endpoints. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

As used herein the term “severe acute respiratory syndrome virus” (SARS-CoV-2) refers to the virus responsible for the Coronavirus disease 2019 (COVID-19) global pandemic. SARS-COV-2 infection is also referred to as “COVID-19” and SARS-CoV-2 is “the virus responsible for COVID-19”. In addition, the current disclosure also encompasses human coronavirus species strains in addition to SARS-COV-2. These include SARS-COV-2 variants, additional SARS-COV strains and the Middle East respiratory syndrome coronavirus (MERS-COV).

As used herein, the term “Spike glycoprotein” or “S protein” (SGP) or (S1) refers to the spike proteins that are anchored in the lipid bilayer of the viral envelope. The lipid bilayer also contains envelope (E) and membrane (M) proteins. The S protein are important in allowing interaction of the coronavirus with the host cell. The peptide of the current disclosure targets the S protein but may also be effective against host cells that interact with the virus via the M and E proteins.

As used herein the term “aquaporin” (AQP) or “water channel” means channel proteins that form pores in the membrane of biological cells, thereby facilitating transport between cells. Aquaporins, help facilitate the movement of water, a polar molecule, in addition to simple diffusion of water. Disruption of normal AQP structure and/or function may be associated with a wide range of diseases and conditions. Disruption in AQP or water channels often manifests as water accumulation and includes, but is not limited to venous insufficiency, heart failure, renal disease, low protein levels, liver problems, deep vein thrombosis, infections, angioedema, certain medications, lymphedema, menstruation, pregnancy, neuromyelitis optica, other viral infections, burn etc.

As used herein, the term “patient” or “subject” as used herein includes human and animal subjects.

As used herein, the term “disorder” is any condition that would benefit from treatment using the peptide of the disclosures. “Disorder” and “condition” are used interchangeably herein and include chronic and acute disorders or diseases, including those pathological conditions that predispose a patient to the disorder in question.

As used herein, the terms “treatment” or “treat” refers to both therapeutic treatment and prophylactic or preventative measures. Those in need of treatment include those having the disorder as well as those prone to have the disorder or those in which the disorder is to be prevented.

The terms “pharmaceutical composition” or “therapeutic composition” or “pharmaceutical formulation” as used herein refers to a compound or composition capable of inducing a desired therapeutic effect when properly administered to a patient.

As used herein, the term “pharmaceutically acceptable carrier” or “physiologically acceptable carrier” refers to one or more formulation materials suitable for accomplishing or enhancing the delivery of the peptide. Such therapeutic or pharmaceutical compositions can comprise a therapeutically effective amount of a peptide, in admixture with a pharmaceutically or physiologically acceptable formulation agent selected for suitability with the mode of administration

Acceptable formulation materials preferably are nontoxic to recipients at the dosages and concentrations employed. The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropylbeta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides preferably sodium or potassium chloride- or mannitol sorbitol), delivery vehicles, diluents, excipients and/or pharmaceutical adjuvants (see, e.g., REMINGTON's PHARMACEUTICAL SCIENCES (18th Ed., A. R. Gennaro, ed., Mack Publishing Company 1990), and subsequent editions of the same, incorporated herein by reference for any purpose).

The optimal pharmaceutical composition will be determined by one of skill in the art, accounting for the intended route of administration, delivery, format, and desired dosage. Aqueous or non-aqueous pharmaceutical compositions and/or vehicles can be used in the current disclosure. Examples of suitable injection vehicles or carriers include but is not limited to water physiological saline solutions such as phosphate buffered saline, and/or serum. Carriers can be supplemented with supplemented as needed with, for examples, vitamins, minerals, or electrolytes. Buffers, including TRIS buffers can be used. Buffers suitable for use have a pH of about 7.0-8.5 (TRIS buffers). The pharmaceutical compositions of the current disclosure can be mixed and prepared for short- or long-term storage using techniques known by those of skill in the art or prepared and used immediately.

As used herein the term “effective amount” and “therapeutically effective amount” when used in reference to a pharmaceutical composition comprising the peptide refers to an amount or dosage sufficient to produce a desired therapeutic result. The effective amount may vary depending on a variety of factors and conditions related to the patient being treated and the severity of the disorder. For example, if the peptide is to be administered in vivo, factors such as the age, weight, and health of the patient as well as dose response curves and toxicity data obtained in preclinical animal work would be among those factors considered. The determination of an effective amount or therapeutically effective amount of a given pharmaceutical composition is well within the ability of those skilled in the art.

### 2. Compositions and Formulations

The disclosure relates to reagents that are peptides each having a specific amino acid sequence. The disclosure also provides compositions, reagents, pharmaceutical formulations comprising such reagents, and methods for preventing and treating viral infections in humans including coronavirus infections, and in particular COVID-19 infections in humans. Further, the disclosure also provides pharmaceutical formulations of such peptide, alone or in combination with suitable excipients and other pharmaceutical formulating agents.

In a preferred embodiment the peptide provided herein has the amino acid sequence MVRIKPASANKPSDD (SEQ ID NO. 1) and is referred to herein as SPIKENET (SPK).

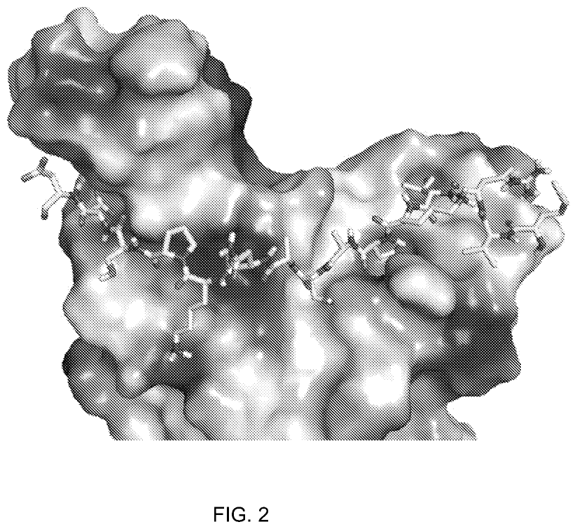

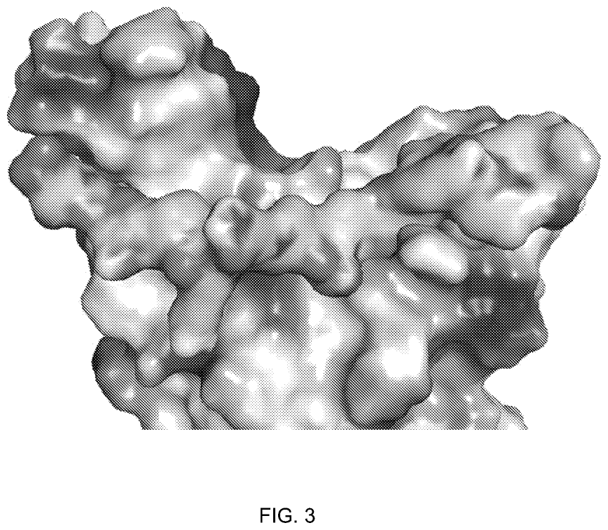

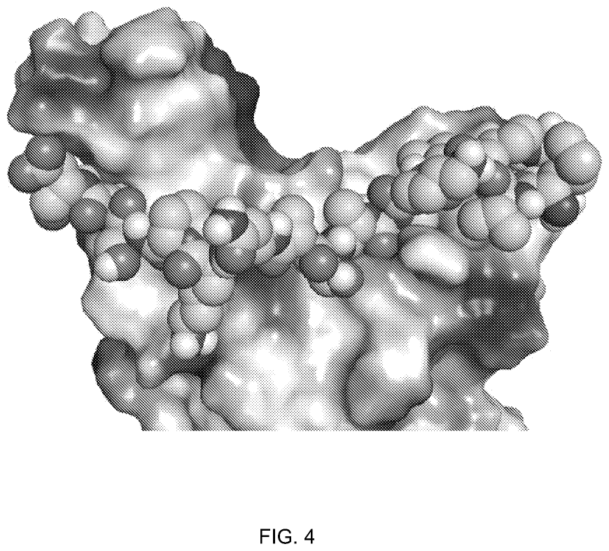

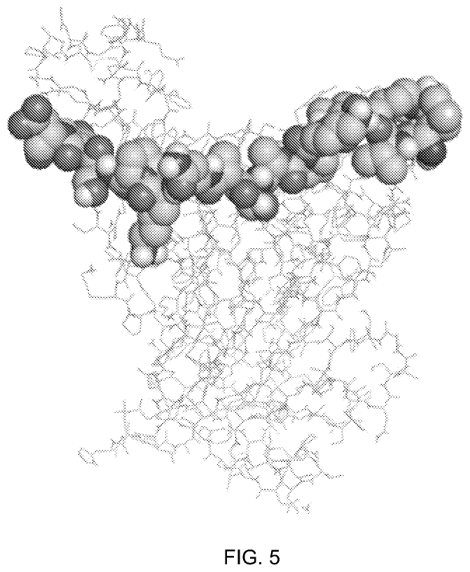

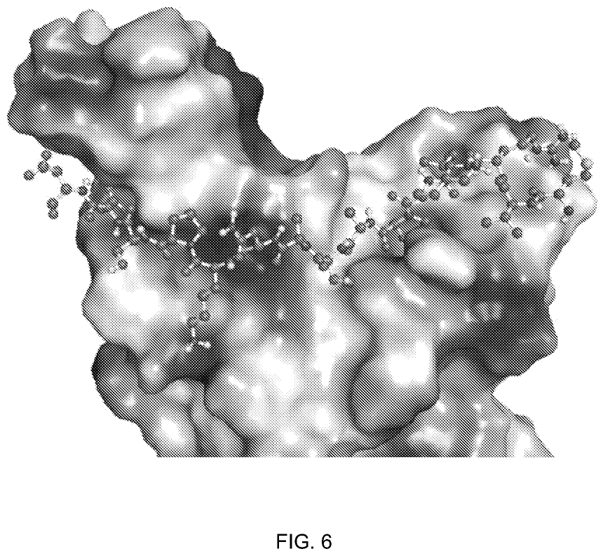

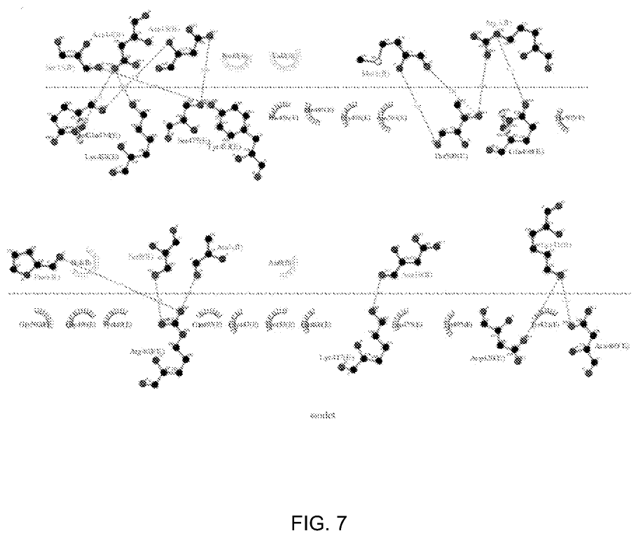

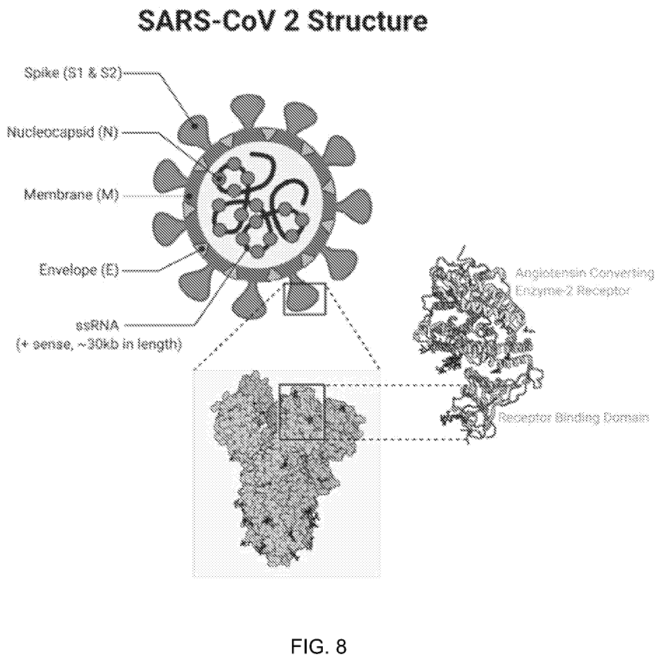

Cluspro computational protein docking program illustrates binding of SPIKENET to the SARS-COV-2 protein SGP. There was highly specific binding affinity of the SPIKENET peptide to the ACE2 binding domain of SGP, shown in FIG. 2. The topography of these interactions is illustrated in variable (to illustrate various advantageous aspects) in FIGS. 3-6. FIG. 7 shows the non-bonded interactions between the binding site residues of the RBD domains and SPIKENET peptide. Consistent with this, SPIKENET is a competitive inhibitor of the spike protein (S1).

This mechanistic binding affinity study by a computational approach described herein has important implications for the management of patients with SARS-CoV-2. The findings represent a new therapeutic approach for treating SARS-COV-2 infection. Thus, the invention provides methods for treating or preventing SARS-COV-2 infection in a human by administering to the human a therapeutically effective amount of a pharmaceutical formulation as provided by the invention. Administration can be achieved by any efficacious manner known to those with skill in the art, including but not limited to aerosol administration to lung.

Methods for synthesizing peptides are known in the art. For example, peptides can be produced using solid phase peptide synthesis on Rink amide resin with Fmoc chemistry and HBTU activation. Resulting resin-bound peptides can then be cleaved and side chains deprotected with TFA/TIPS/H₂O (95:2.5:2.5) and precipitated with cold ether. Crude peptides can be purified by RP-HPLC to −98% purity). Purified peptides can be confirmed by mass spectrometry (MS) on a Bruker Esquire Ion Trap electrospray mass spectrometer, and peptide stocks lyophilized for storage at −20° C. Peptide stocks can then be thawed and reconstituted in filtered deionized distilled water to a concentration of 2 mg/mL. See, for example, Stowikowski, M. & Fields, G. B. Introduction to Peptide Synthesis. Curr. Protoc. Prot. Sci. Unit-18.1 (2002; incorporated by reference).

In an alternate method, peptides can be synthesized by standard solid phase peptide synthesis technique on Wang resin using F-moc chemistry and HBTU activation. Automated peptide synthesis, for example the CSBio 336s (CSBio, USA) automated peptide synthesizer, can be used for the synthesis. The resulting resin-bound peptides can then be cleaved and side-chain-deprotected using Reagent K (trifluoroacetic acid/thioanisole/H₂O/phenol/ethanedithiol (87.5:5:5:2.5)) and, precipitated by cold ether. The crude peptides obtained are advantageously purified by reverse phase high performance liquid chromatography up to a >98% purity (Gemini 10 .mu. C18 110A column). The masses of the purified peptides are checked by mass spectroscopy using a MALDI-TOF mass spectrometer. One of skill in the art will recognize additional techniques can be used to generate the peptides disclosed.

SPIKENET has shown highly specific binding affinity to the ACE2 binding domain of spike glycoprotein (S1) as well as efficacy for use in both in vitro and in vivo. In addition, SPIKENET can effectively prevent SARS-COV-2 endocytosis. SPIKENET has broader uses as a potent agent to treat any disease that display severe oxidative stress related to SPIKENET's protective effect against various toxins induced generation of free radicals. Accordingly, SPIKENET can be used to treat cancer, Alzheimer's disease, Parkinson's disease, Diabetes, cardiovascular conditions including, but not limited to hypertension, atherosclerosis, stroke, asthma, and male infertility. SPIKENET is also protective against multi-organ failure, and as such can be used for many life-threatening conditions that exhibit multi-organ failure such as sepsis, systemic inflammatory response syndrome, GI bleeding, kidney diseases and respiratory failure, graft versus host disease, radiation injury. The Neuroprotective effects of SPIKENET make it an ideal candidate to treat many neurological conditions: these include ischemic brain injury, traumatic brain injury, perinatal brain injury, various encephalopathies, Alzheimer's disease, neurodegenerative diseases, autism spectrum disease, neuromuscular disorders.

In further aspects of the disclosure are compositions and/or pharmaceutical formulation comprising a therapeutically effective amount of a peptide and one or a plurality of excipients, adjuvants, or other formulation components for treating or preventing coronavirus infection, in a subject. In certain embodiments, the formulation comprises peptides having an amino acid sequence comprising:

(SEQ ID NO. 20)
(M)nVR(I/L)KP(G/A)(S/T)(A/G)NKP(S/T)(D/E)D,

wherein n=0 or 1, wherein the peptide does not have the amino acid sequence MVRIKPGSANKPSDD (SEQ ID NO: 21) and wherein variant amino acid sequence residues are set forth in the alternative at each substituted position. In other aspects, the pharmaceutical formulations comprise variant peptides having an amino acid sequence comprising:
MVRIKPASANKPSDD (SEQ ID NO: 1); MVRIKPGTANKPSDD (SEQ ID NO: 2); MVRIKPGTANKPTDD (SEQ ID NO: 3); MVRIKPGSGNKPSDD (SEQ ID NO: 4); MVRIKPATANKPSDD (SEQ ID NO: 5); MVRIKPGTGNKPSDD (SEQ ID NO: 6); MVRIKPGTGNKPTDD (SEQ ID NO: 7); MVRIKPATANKPTDD (SEQ ID NO: 8); MVRIKPATGNKPTDD (SEQ ID NO: 9); MVRIKPATGNKPSDD (SEQ ID NO: 10); VRLKPASANKPSED (SEQ ID NO: 11); VRLKPASGNKPSED (SEQ ID NO: 12); VRLKPGSGNKPSED (SEQ ID NO: 13); VRLKPATGNKPTED (SEQ ID NO: 14); VRLKPGTANKPTED (SEQ ID NO: 15); VRLKPATANKPTED (SEQ ID NO: 16). VRLKPGTGNKPTED (SEQ ID NO: 17); VRLKPGTANKPSED (SEQ ID NO: 18); or VRIKPGTANKPSED (SEQ ID NO: 19) or salts or derivatives thereof. The variants described herein could be used to treat infection or sequelae thereof relating to impairment of skeletal, muscular, nervous, endocrine, cardiovascular, lymphatic, reparatory, digestive, urinary, or reproductive function.

### 3. Methods of Treatment

Methods for using such pharmaceutical formulations are provided for treating and preventing viral disease. More specifically in one embodiment is a method of treating or preventing infection of a human with a coronavirus, the method comprising administering to a human in need thereof a pharmaceutical formulation containing SPIKENET. The peptide can be administered via a variety of routes including, but not limited to, intranasally, intravenously, mucosally, orally, subcutaneously, or intramuscularly.

SPIKENET dosing is disease and patient specific but can be administered up to four times per day. More specifically SPIKENET can be administered once per day, twice per day, three times per day, or four times per 24-hour period. A dosing schedule that accounts for the total SPIKENET administration and frequency of administration can be established after determination of effective dose for a given condition. Effective doses of SPIKENET can range from 0.5 mg/kg-10 mg/kg body weight.

SPIKENET can be used in numerous conditions as described herein that exhibit severe oxidative stress, edema, LPS-mediated pathologies, inflammation, and/or thrombosis or coagulation.

SPIKENET administration exhibits a significant technical effect in treating edema and progressive water accumulation in tissues. The inventors have found that, unexpectedly, SPIKENET administration significantly reduces organ edema making it an ideal peptide for use in wide-ranging conditions that exhibit similar disease progression mediating water accumulation. These conditions may include, but are not limited to venous insufficiency, heart failure, renal disease, low protein levels, liver problems, deep vein thrombosis, infections, angioedema, certain medications, lymphedema, menstruation, pregnancy, neuromyelitis optica, other viral infections, and burns.

Further unexpectedly SPIKENET demonstrates a potent effect in preventing lipopolysaccharide mediated pathophysiological alterations. Accordingly, SPIKENET administration can be extended to treat various inflammatory and autoimmune diseases. These include fatty liver disease, endometriosis, Type 2 diabetes mellitus, Type 1 diabetes mellitus, Inflammatory bowel disease (IBD), asthma, rheumatoid arthritis, obesity, SLE, antiphospholipid antibody syndrome, malignancy, etc. SPIKENET may further be helpful in preventing or treating pregnancy complications, such as pregnancy loss, implantation failure, fetal growth restriction, preeclampsia, gestational diabetes, preterm birth, abruption, preterm rupture of membranes, cholestasis of pregnancy, ovarian hyperstimulation syndrome, thrombosis associated with pregnancy.

Also, unexpectedly the inventors demonstrate that SPIKENET prevented thrombosis induced by MHV-1 virus. This supports SPIKENET as an effective peptide in addressing coagulation and conditions caused by coagulation issues. These includes, but is not limited to, hemophilia, von Willebrand disease, clotting factor deficiencies, idiopathic thrombocytopenia purpura, hypercoagulable states, deep venous thrombosis, thrombotic thrombocytopeniarpura, malignancy, disseminated intravascular coagulation etc.

Pharmaceutical compositions of the invention to be used for in vivo administration typically must be sterile. This can be accomplished by use of multiple methods, as known by one of skill in the art. Pharmaceutical compositions can also be is lyophilized and sterilized or sterilized then lyophilized. Pharmaceutical compositions for storage can be stored as solutions, suspensions, gels, emulsions, solids, or as a lyophilized powder.

The effective amount of a peptide composition to be employed therapeutically varies depending on the indication, route of administration, and body size. A typical dosage can range from about 0.005 mg/kg (5 μg/kg)-10,000 mg/kg accounting for the factors noted above. More specifically the typical dose can range from about 0.01 mg/kg g to about 10,000 mg/kg; about 0.1 mg/kg to about 10,000 mg/kg; about 1.0 mg/kg to about 10,000 mg/kg; about 10 mg/kg to about 10,000 mg/kg; about 100 mg/kg to about 10,000 mg/kg; about 250 mg/kg to about 10,000 mg/kg; about 500 mg/kg to about 10,000 mg/kg; about 1000 mg/kg to about 10,000 mg/kg; about 1500 mg/kg to about 10,000 mg/kg; about 2000 mg/kg to about 10,000 mg/kg; about 2500 mg/kg to about 10,000 mg/kg; about 3000 mg/kg to about 10,000 mg/kg; about 3500 mg/kg to about 10,000 mg/kg; about 4000 mg/kg to about 10,000 mg/kg; about 4500 mg/kg to about 10,000 mg/kg; about 5000 mg/kg to about 10,000 mg/kg; about 5500 mg/kg to about 10,000 mg/kg; about 6000 mg/kg to about 10,000 mg/kg; about 6500 mg/kg to about 10,000 mg/kg; about 7000 mg/kg to about 10,000 mg/kg; about 7500 mg/kg to about 10,000 mg/kg; or about 8000 mg/kg to about 10,000 mg/kg.

In further aspects the typical dose can range from about 0.005 mg/kg to about 10,000 mg/kg; about 0.005 mg/kg to about 5000 mg/kg; about 0.005 mg/kg to about 2500 mg/kg; about 0.005 mg/kg to about 2000 mg/kg; about 0.005 mg/kg to about 1500 mg/kg; about 0.005 mg/kg to about 1000 mg/kg; about 0.005 mg/kg to about 500 mg/kg; about 0.005 mg/kg to about 250 mg/kg; about 0.005 mg/kg to about 200 mg/kg; about 0.005 mg/kg to about 150 mg/kg; about 0.005 mg/kg to about 100 mg/kg; about 0.005 mg/kg to about 50 mg/kg; about 0.005 mg/kg to about 25 mg/kg; about 0.005 mg/kg to about 24 mg/kg; about 0.005 mg/kg to about 23 mg/kg; about 0.005 mg/kg to about 22 mg/kg; about 0.005 mg/kg to about 21 mg/kg; about 0.005 mg/kg to about 20 mg/kg; about 0.005 mg/kg to about 19 mg/kg; about 0.005 mg/kg to about 18 mg/kg; about 0.005 mg/kg to about 17 mg/kg; about 0.005 mg/kg to about 16 mg/kg; about 0.005 mg/kg to about 15 mg/kg; about 0.005 mg/kg to about 14 mg/kg; about 0.005 mg/kg to about 13 mg/kg; about 0.005 mg/kg to about 12 mg/kg; about 0.005 mg/kg to about 11 mg/kg; about 0.005 mg/kg to about 10 mg/kg; about 0.005 mg/kg to about 9 mg/kg; about 0.005 mg/kg to about 8 mg/kg; about 0.005 mg/kg to about 7 mg/kg; about 0.005 mg/kg to about 6 mg/kg; or about 0.005 mg/kg to about 5 mg/kg; about 0.005 mg/kg to about 4 mg/kg; about 0.005 mg/kg to about 3 mg/kg; about 0.005 mg/kg to about 2 mg/kg; or about 0.005 mg/kg to about 1 mg/kg.

In further aspect the typical dose can range from about 0.005 mg/kg to about 10,000 mg/kg; about 0.1 mg/kg to about 5000 mg/kg; about 0.1 mg/kg to about 5000 mg/kg; about 0.1 mg/kg to about 2500 mg/kg; about 0.1 mg/kg to about 2000 mg/kg; about 0.1 mg/kg to about 1500 mg/kg; about 0.1 mg/kg to about 1000 mg/kg; about 0.1 mg/kg to about 500 mg/kg; about 0.1 mg/kg to about 250 mg/kg; about 0.1 mg/kg to about 200 mg/kg; about 0.1 mg/kg to about 150 mg/kg; about 0.1 mg/kg to about 100 mg/kg; about 0.1 mg/kg to about 50 mg/kg; about 0.1 mg/kg to about 25 mg/kg; about 0.1 mg/kg to about 20 mg/kg; or about 0.1 mg/kg to about 10 mg/kg.

Dosing frequency will depend upon the pharmacokinetic parameters of the peptide with consideration of the disease. The therapeutic composition can be administered as a single dose or in two or more doses which may contain the same or different amount of the peptide at any time during a 24-window. Dosing parameters can be established using dose-response curves as known by one of skill in the art.

The peptide and/or pharmaceutical composition can be administered using known methods and injection routes such as intravenous, mucosally, orally, intraperitoneal, intracerebral (intraparenchymal), intracerebroventricular, intramuscular, intraocular, intraarterial, intraportal, or intralesional routes; by sustained release systems. In a preferred embodiment the peptide and/or pharmaceutical composition is administered intranasally.

Animal models include and MHV-1 SARS-COV-2 model in which MHV-1 infected A/J mice exhibit clinical SARS-like disease with high mortality as described in the examples below. A K18-hACE2 transgenic mouse is also disclosed herein that will be examined to recapitulate events occurring in humans associated with SARS COV-2 infection thereby confirming the efficacy of the MVH-1 SARS-COV-2 model.

Having described the invention in detail and by reference to specific aspects and/or embodiments thereof, it will be apparent that modifications and variations are possible without departing from the scope of the invention defined in the appended claims. More specifically, although some aspects of the present invention can be identified herein as particularly advantageous, it is contemplated that the present invention is not limited to these particular aspects of the invention.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps.

### EXAMPLES

The Examples that follow are illustrative of specific embodiments of the disclosure, and various uses thereof. They are set forth for explanatory purposes only and should not be construed as limiting the scope of the disclosure in any way.

### General Materials and Methods

### Mice

Female A/J mice (8 weeks of age, ˜22 g) were maintained in micro-isolated cages (paired in a single cage) and fed a standard lab chow diet and water ad libitum. Mice were divided into three groups: healthy controls; healthy controls inoculated with Dulbecco's Modified Eagle Medium (DMEM) alone, intranasal; and intranasal MHV-inoculated mice.

### Viral Inoculation

MHV-1 was purchased from American Type Culture Collection (ATCC, cat #VR-261, Manassas, VA). Mice were inoculated with 5,000 PFU intranasally [Williams et al., (2021) Forensic Sci. Int.; Bermejo et al., (2004) Viral Immunol. Briefly, 5×10³ PFU MHV-1 was mixed with 50 μl of ice-cold Dulbecco's modified Eagle's medium and instilled into the nares immediately, and mice were observed until the virus was inhaled.

### Scoring

Control mice and mice challenged with MHV-1 were monitored for clinical signs of infection. Symptoms were scored as: 0) no symptoms, I) drowsy and lack of movement, II) slightly ruffled fur and altered hind limb posture, III) ruffled fur and mildly labored breathing, IV) ruffled fur, inactive, moderately labored breathing, V) ruffled fur, obviously labored breathing, and lethargy, and VI) moribund and death.

### Clinical Observation

Mice with a disease stage of V-VI (occurring at 7-12 days) were weighed, euthanized, and lungs, liver, kidney, heart, and brain removed and fixed in 10% formalin, processed routinely for paraffin sections, and stained with Hematoxylin and Eosin. To ascertain the extent of liver failure, blood was collected via cardiac puncture, and serum was used to measure liver enzymes. Levels of aspartate aminotransferase (AST), alanine aminotransferase (ALT), alkaline phosphatase (ALP), and bilirubin content were determined at the onset of the disease stages of V-VI, as previously described (Litwack et al., (2018) Human Biochemistry; Cai, et al. (2020) J. Hepatol). Body weight was measured daily.

### Examination of Oxidative Stress Markers and AQPs in Tissues by Immunohistochemistry

Paraffin embedded tissue sections from healthy control and MHV-1 virus exposed mice (10 micron) were incubated with antibodies specific to 4-HNE and MDA, as well as AQPs (AQP1, 4, 5, and 8). Sections were washed and incubated in respective horseradish peroxidase-conjugated secondary antibodies. Immuno-fluorescent images were acquired with Nikon A1R Laser Scanning Confocal and TIRF combined with advanced 3D reconstruction software (Imaris).

### Statistical Analysis

Data were subjected to analysis of variance followed by Tukey's multiple comparison test. A statistical analysis showing p<0.05 was considered significant.

### Example 1. Modeling Studies

Protocol: the effect of SPIKENET on SARS-COV-2 Spike protein was tested with Cluspro computational protein docking program. Initial structures of the MHV1-NTD domain and CCM human receptor were obtained from the PDB database from the structure of 6vsj, and the NTD domain truncated at residues 16-283 from MHV1-SP for dynamic study. The ligand peptide of SPK has 15 residues with the sequence of MVRIKPASANKPSDD (SEQ ID NO: 1; SPIKENET) and its 2D structure built using the Avogadro (v. 1.2.0) molecular modeling program, and geometrical optimization performed using the Steepest Descent algorithm with the force field of UFF to get energy-optimized 3D structure.

SPIKENET demonstrated high binding affinity for the ACE2 binding domain of the spike glycoprotein (SGP).

### Example 2. In Vitro SPIKENET Studies

Protocol (Astrocyte Culture): In order to examine if SPIKENET has any protective effect in a toxicity model using an in vitro system, we utilized a primary central nervous system cell culture system. Briefly, primary cultures of cortical astrocytes were prepared by mincing and homogenizing brain cerebral cortices of 1-2-day old rat pups. Homogenate was filtered and the pellet re-suspended and seeded onto 35 mm culture dishes in Dulbecco's modified Eagle's medium containing penicillin, streptomycin and 15% fetal bovine serum. Culture plates were incubated at 37° C. with 5% CO2 and 95% air. Culture media was changed twice weekly. On day 10 post-seeding, fetal bovine serum was replaced with 10% horse serum. After 14 days, cultures were treated with 0.5 mM dibutyryl cAMP (Sigma, St. Louis, MO, USA) to enhance cellular differentiation (2). Cultures consisted of at least 95% astrocytes as determined by glial fibrillary acidic protein (GFAP) immunohistochemistry. All cultures used were 24-28 days old. (Ducis I, Norenberg L O, Norenberg M D (1989) Effect of ammonium chloride on the astrocyte benzodiazepine receptor. Brain Res 493(2):362-365; Juurlink B H, Hertz L (1985) Plasticity of astrocytes in primary cultures: an experimental tool and a reason for methodological caution. Dev Neurosci 7(5-6):263-277).

Microglial primary cultures were grown on a monolayer of astrocyte cultures prepared from brains of 1-day old pups. Cerebral cortices were dissected, stripped of meninges, and were minced in Hank's balanced salt solution (0.137 M NaCl, 0.2 M NaH₂PO₄, 0.2 M KH₂PO₄, 5.4 mM KCl, 5 mM glucose, 58.4 mM sucrose, 0.25 μg/ml Fungizone, and 1×10⁶ U penicillin/streptomycin), to which 0.25% trypsin was added and incubated for 30 min at 37° C. Trypsin reaction was stopped by adding 5 ml of DMEM containing 10% fetal bovine serum (FBS) and 1% penicillin/streptomycin. The suspension was triturated several times. The mixed brain cell suspension was then passed through sterile filters (130, 40 μm). Final cell suspension was seeded into T75 flasks and allowed to grow for 4 days, medium was then changed, and incubation continued for an additional 6 days. Microglia were harvested from cortical cultures by shaking the flasks on an orbital shaker (100 rpm) for 1 h at 37° C. Media containing microglia was collected, centrifuged, and replated at a density of 1×10⁴ cells in 35 mm² plates. Cultures were at least 98% microglia as determined by ED1 immunoreactivity. (Flanary B E, Streit W J (2004) Progressive telomere shortening occurs in cultured rat microglia, but not astrocytes. Glia 45(1):75-88).

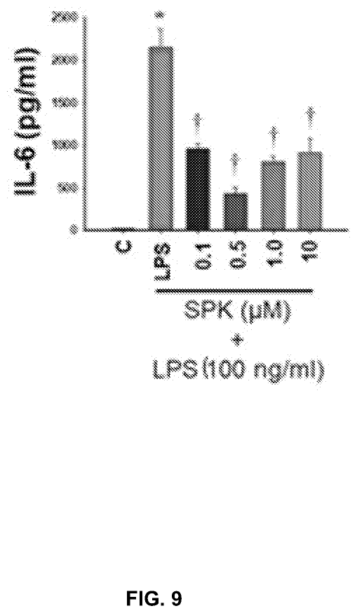

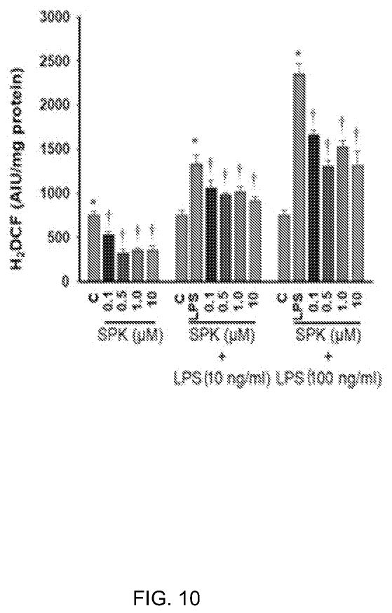

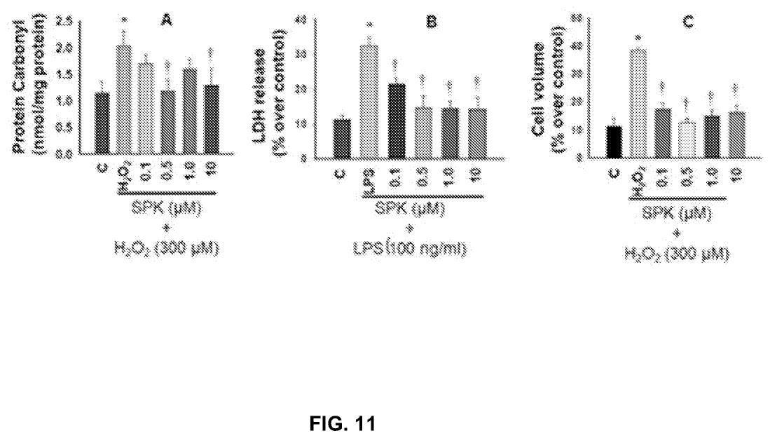

SPIKENET (at 100 nM-10 UM concentrations) diminished lipopolysaccharide (LPS)-induced free radical formation, cytokine (IL-6) release and hydrogen peroxide induced protein carbonyls in primary cultures of rat brain microglia. LPS-induced LDH release was inhibited by SPIKENET in primary cultures of rat brain microglia, as well as hydrogen peroxide induced cell swelling in primary cultures of rat brain astrocytes (See FIGS. 9-11).

Conclusion: Lung edema and its contribution to respiratory insufficiency are major events in SARS COV-2 infection, SPIKENET due to its non-toxic, immune modulatory, antioxidant properties, as well as its effectiveness in reducing edema development, is useful in mitigating SARS-COV-2-induced infection in humans.

### Example 3. SPIKENET Binding Affinity

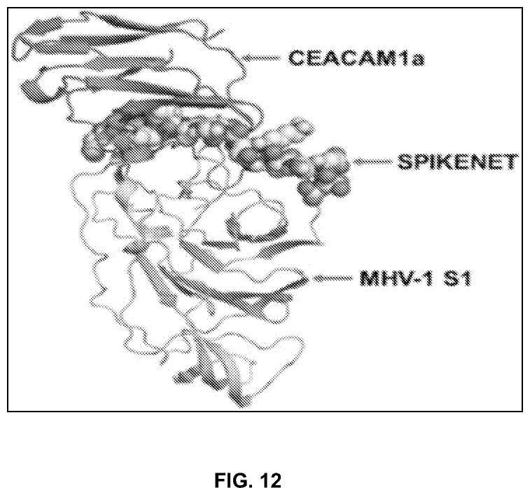

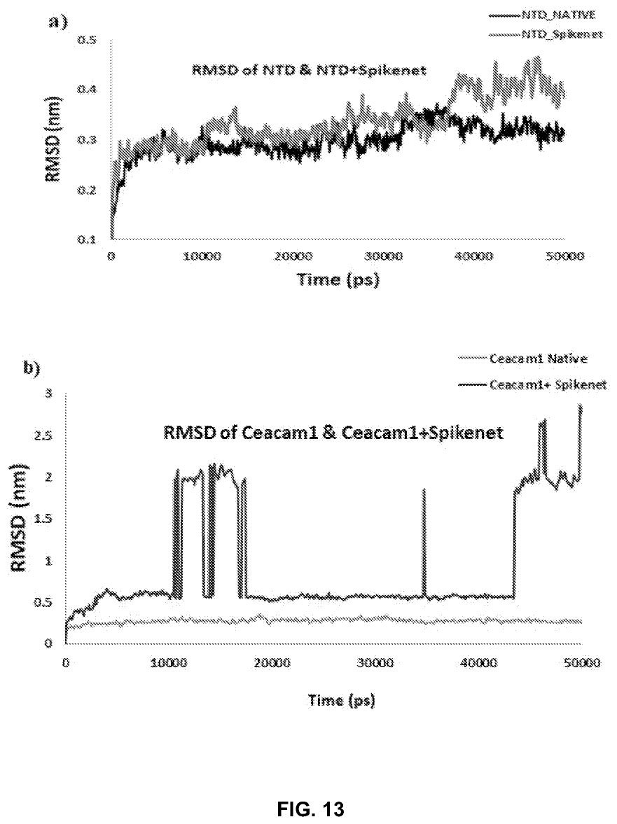

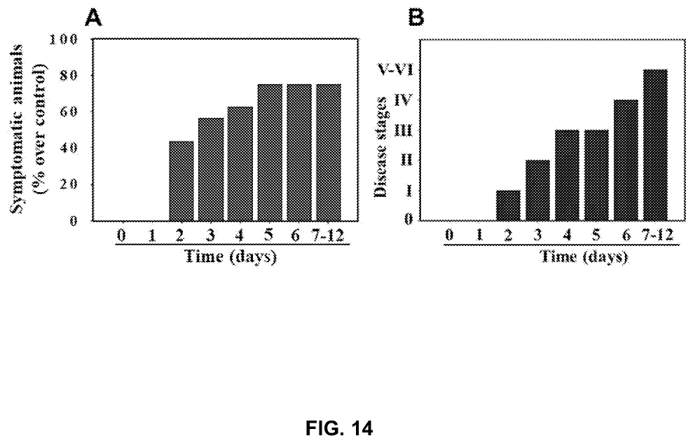

Protocol: Spike protein (SPK) binding affinity to the CEACAME1a (CCM) binding domain of the MHV-1-S1 was assessed by modelling (FIG. 12), as well as by molecular dynamic studies (FIGS. 13a and 13b).

The binding efficacy of SPK peptide with NTD domain of MHV1-S1 and receptor CCM was assessed. The truncated NTD and CCM domains were docked with SPK peptide using ClusPro server at the NTD and CCM binding site. SPK binding affinity with NTD and CCM was subjected to Molecular dynamics (MD) simulation to determine binding affinity of SPK with NTD and CCM. MD simulation was performed with the default setting of the GROMACS 5.4.1 package and simulation done by applying GROMOS96 54a7 force field for all protein atoms. The simulation was performed three times for every target with the similar initial configurations, and identical results were obtained. Targets with SPK complexes were immersed in a cubic box with water as the solvent. To neutralize the system, respective Na+/Cl− ions were included as shown in the table; each system was subjected to energy minimization for 50000 steps by steepest descents

TABLE 1
Na⁺/Cl⁻ ions used in Binding Efficacy Studies

| | Residues | No. of | No. of | Size |
|---|---|---|---|---|
| | range | the water | ions for | of the |
| Target | system | molecule | equilibration | system |
| NTD | 16 to 283 | 17834 | 4Cl⁻ | 83.506 Å |
| NTD + Spikenet | 16 to 283 | 19955 | 5Cl⁻ | 86.703 Å |
| Ceacam1 | 35 to 141 | 8211 | 3Na⁺ | 64.557 Å |
| Ceacam1 + Spikenet | 35 to 141 | 9884 | 2Na⁺ | 68.496 Å |

After minimization of complex structure, position restrained MD simulation was done upon slow heating to 300K in NVT and NPT ensemble with a constant particles number, constant temperature, constant volume, and constant pressure, respectively, at 1 atm pressure throughout 500 ps. The final production MD calculation was then carried out for a total of 50 ns MD simulation for our target complexes of NTD+SPK and CCM+SPK with a time step of 1fs at the constant pressure (1 atm) and temperature (300 k). The MD trajectories were analyzed from time to time using the VMD program.

Trajectories of both complexes, NTD-SPK and CCM-SPK in MD analysis were analyzed to understand the binding affinity of SPK with NTD/CCM domains and their conformational changes during the simulation. Consistent with this, the root mean square deviation (RMSD) for protein backbone atoms using least-squares fitting, and the root mean square fluctuation for every residue were calculated for both complexes and the native proteins using their final coordinates obtained from MD simulation. The RMSD of both complexes with their respective native proteins are shown in FIGS. 13A & 13B. Comparing the RMSD of both NTD/NTD+SPK (FIG. 13A) after 50 ns dynamic simulation, the structures showed complete equilibration in the system. The SPK peptide is well stabilized with high affinity at the CCM binding location of NTD. However, RMSD analysis of CCM and CCM+SPK structures after the 50 ns dynamic simulation exhibited more flexibility at the NTD binding site of CCM than native CCM (FIG. 13B), suggesting SPK peptide detachment/displacement over CCM. The initial detachment of SPK peptide showed in the range of 10 to 18 ns and after 45 ns SPK peptide has completely dissociated from the CCM.

The stabilized RMSD of NTD with SPK demonstrated the reliability of peptide binding with the NTD domain. Similar affinity was also identified with SP of other variants of SARS-COV-2, strongly supporting the use of SPK to prevent COVID-19.

### Example 4. Animal Model Pre-Clinical Studies

Protocol: Multi-organ failure is the major aspect in SARS-COV-2 infection and associated with subsequent death. Mice were examined to determine if exposure to Murine Hepatitis Virus Strain-1 (MHV-1) produced symptoms in mice similar to that of COVID-19 in humans. Organ integrity was examined, with and without SPK.

Female A/J mice (8 weeks of age, ˜22 g) were maintained in micro-isolated cages (paired in a single cage) and fed a standard lab chow diet and water ad libitum. Mice were divided into three groups: healthy non-infusion controls; healthy infusion controls (infused with DMEM); or intranasal infusion of MHV-1.

MHV-1 was purchased from American Type Culture Collection (ATCC, cat #VR-261, Manassas, VA). Mice were inoculated with 5,000 PFU intranasally [Williams et al., (2021) Forensic Sci. Int.; Bermejo et al., (2004)]. Approximately, 5×10³ PFU MHV-1 were mixed with 50 μl of ice-cold Dulbecco's modified Eagle's medium and instilled into the nares immediately, and mice observed until the virus was inhaled.

Control mice and mice challenged with MHV-1 were monitored for clinical signs of infection and scored accordingly. Symptoms were scored as: 0) no symptoms, I) drowsy and lack of movement, II) slightly ruffled fur and altered hind limb posture, III) ruffled fur and mildly labored breathing, IV) ruffled fur, inactive, moderately labored breathing, V) ruffled fur, obviously labored breathing and lethargy, and VI) moribund and death.

Mice with disease stage symptoms scored as V-VI (occurring at 7-12 days) were weighed, euthanized, and lungs, liver, kidney, heart, and brain removed and fixed in 10% formalin, processed routinely for paraffin sections and stained with Hematoxylin and Eosin. To ascertain the extent of liver failure, blood was collected via cardiac puncture, and serum was used to measure liver enzymes. Levels of aspartate aminotransferase (AST), alanine aminotransferase (ALT), alkaline phosphatase (ALP), and bilirubin content were determined at the onset of the disease stages scored as V-VI, as previously described (Litwack et al., (2018) Human Biochemistry, Cai, et al. (2020) J. Hepatol). Body weight was measured daily.

Paraffin embedded tissue were examined for presence of viral proteins and AQPs in tissues. Tissue sections from healthy control and MHV-1 virus exposed mice (10 micron) were incubated with antibodies specific to S1, nucleocapsid, CCM, TRPMSS2, as well as AQPs (AQP0-12). Sections were washed and incubated in respective horseradish peroxidase-conjugated secondary antibodies. Immuno-fluorescent images were acquired with Nikon A1R Laser Scanning Confocal and TIRF combined with advanced 3D reconstruction software (Imaris).

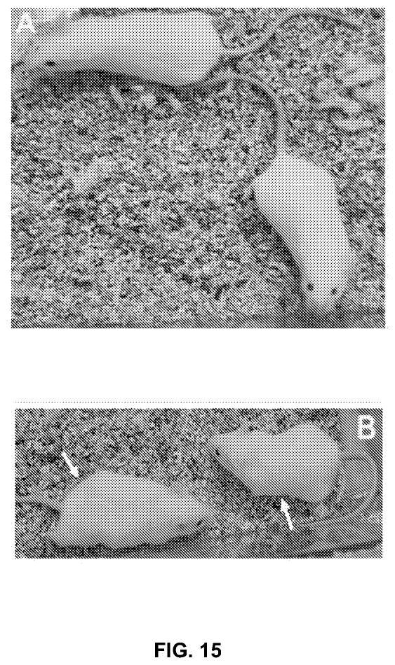

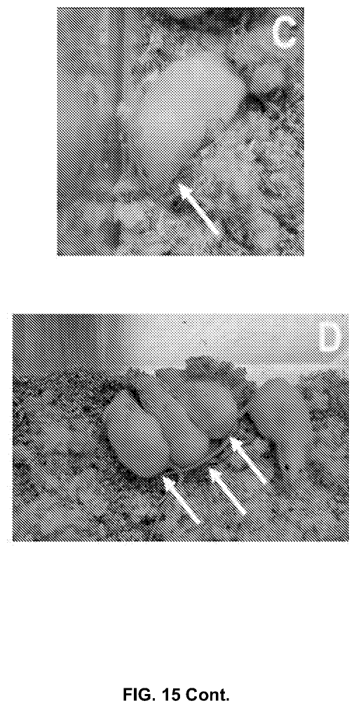

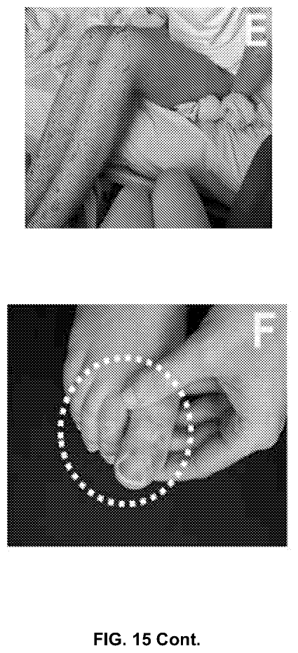

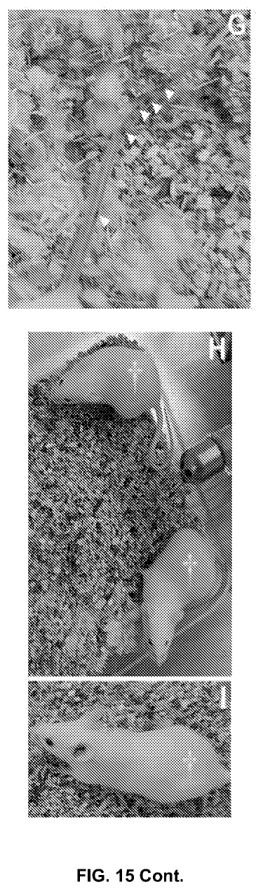

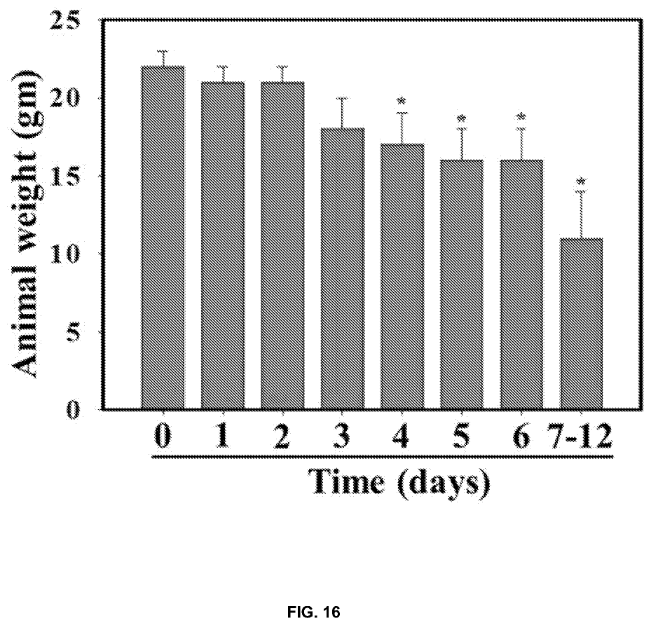

MHV-1 inoculated mice experienced death and weight loss (FIG. 15 and FIG. 16). MHV-1-infected mice at days 7-8 exhibited severe lung inflammation, peribronchiolar interstitial infiltration, bronchiolar epithelial cell necrosis and intra-alveolar necrotic debris, alveolar exudation (surrounding alveolar walls have capillaries that are dilated and filled with RBCs), mononuclear cell infiltration, hyaline membrane formation, the presence of hemosiderin-laden macrophages, as well as interstitial edema. At death, lungs demonstrate severe interstitial pneumonitis with large areas of complete consolidation of the lungs. The interstitial inflammatory reaction includes hyaline membranes, fibrin deposition, and heavy lymphocyte and macrophage infiltrates. Infiltrating cells in MHV-1-infected lung tissue from A/J mice related to macrophage and neutrophil presence on days 2-6 post MHV-1 infection. T cells are also present in the infiltrate by day 6 post-infection. Livers of infected mice show normal histology following infection but then the presence of severe hepatic congestion similar to humans.

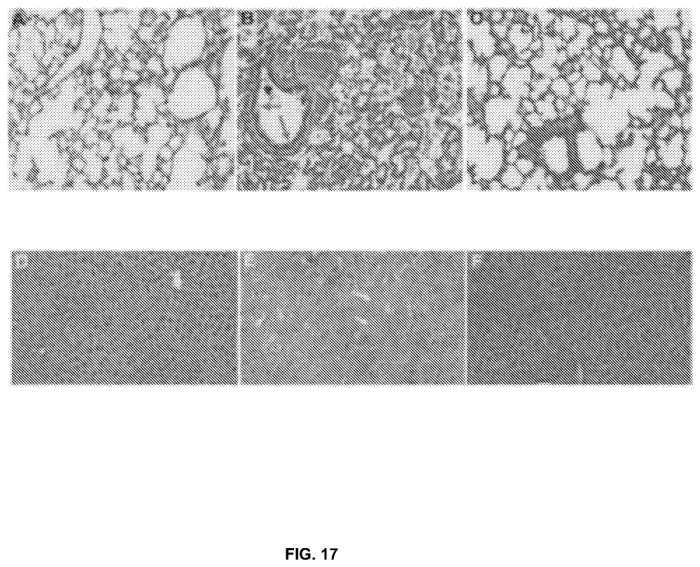

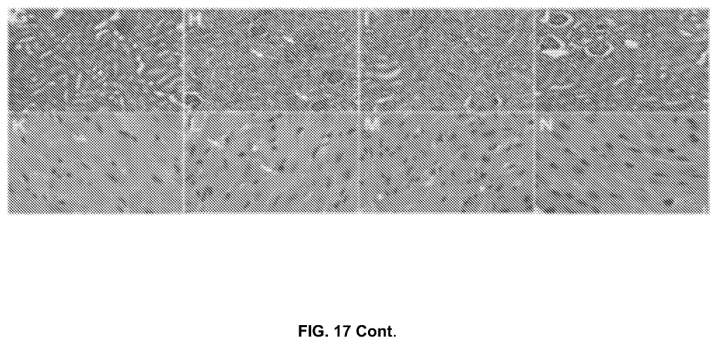

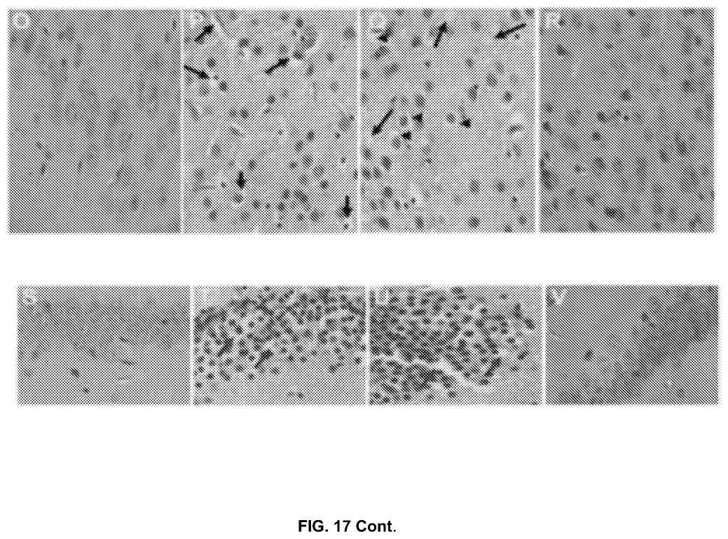

When compared to control-uninfected mice, infected mice showed severe liver vascular congestion, luminal thrombosis of portal and sinusoidal vessels, hepatocytes degeneration, cells necrosis and hemorrhagic changes. Proximal and distal tubular necrosis, hemorrhage in interstitial tissue, and vacuolation of renal tubules were observed in the kidney. The heart was characterized by severe interstitial edema, vascular congestion and dilation, and red blood cells extravasation into interstitium. The brain of MHV-1 infected mice brain exhibited congested blood vessels, perivascular cavitation, cortical pericellular halos, vacuolation of neuropils, darkly stained nuclei, pyknotic nuclei and associated vacuolation of the neuropil in the cortex, and acute eosinophilic necrosis and necrotic neurons with fragmented nuclei and vacuolation in the hippocampus (FIG. 17).

These experimented showed that A/J mice were highly susceptible to MHV-1-induced pulmonary disease via following intranasal infection. These findings suggested widespread thrombotic events seen in a highly relevant and surrogate animal model for SARS-COV-2 mimics reported findings in SARS-COV-2-infected humans and represents an attractive, safer, animal model to study SARS-COV-2 infection, pathophysiologic mechanisms. The results further demonstrated that SPK provides a potential therapeutic intervention.

### Example 5. Liver Enzymes in an Animal Model

Protocol: The status of liver enzymes and liver enzymes (AST and ALT) were determined in the same mice, as noted above.

These experiments showed that increased liver enzyme (AST and ALT) expression was present in MHV-1-infected mice, similar to that observed in drug or chemical induced acute liver failure, and characteristic of COVID-19 patients. AST levels increased over 30-fold in MHV-1 inoculated mice, as compared to un-inoculated mice (3459.2±684.1 units/l in MHV-1 infected mice, as compared to 96.8±14.2 units/l in control group; 34.7-fold increase over control). MHV-1 infected mice also displayed a greater than 90-fold increase in ALT level (3068.5±861.3 units/l in MHV-1 infected mice, as compared to 31.5±11.6 in control group; 96.4-fold increase over control). ALP and bilirubin levels were also increased in MHV-1 infected mice (986.3±178.4 units/l of ALP in MHV-1 infected mice, as compared to 589.1±108.7 in control group; 67% increase over control; and 0.86±0.2 mg/L of bilirubin, as compared to 0.075±0.02 mg/l in uninfected mice; 10.4-fold increase over control) (Table 2). Further, exposure of mice with DMEM had no effect on alterations in liver enzymes (i.e., levels are identical to that of healthy controls), while treatment of these mice with 5 mg/kg SPIKENET ameliorated the enzyme levels (Table 2). These findings strongly suggest that MHV-1 leads to severe liver injury, which was similar to that observed in patients with SARS-COV-2 infection.

TABLE 2
AST and ALT Levels in MHV-1 infected and control mice

| | Control | MHV-1 | MHV-1 + SPIKENET |
|---|---|---|---|
| AST | 96.8 ± 14.2 | 3459.2 ± 684.1* | 608.7 ± 356.8^{†} |
| (units/l) | | | |
| ALT | 31.5 ± 11.6 | 3068.5 ± 861.3* | 1029.1 ± 436.7^{†} |
| (units/l) | | | |

Mean values ± SD.
*Statistically significant difference from control (p < 0.05);
^{†}P < 0.05, different from MHV-1.

### Example 6. Oxidative Stress With or Without SPIKENET

Protocol: A hallmark of SARS-COV-2 infection is systemic proinflammation. Inflammatory disease associated with the production of reactive oxygen species (ROS) followed by oxidative stress (OS). Lipid peroxidation-derived aldehydes, 4-hydroxynonenol (4-HNE)/malondialdehyde (MDA) were identified in various organs of MHV-1 inoculated mice (6 days post-MHV-1) using confocal microscopy and analysis as described above in order to determine the involvement of oxidative stress in SARS-CoV-2 infection and its impact on pathophysiologic alterations.

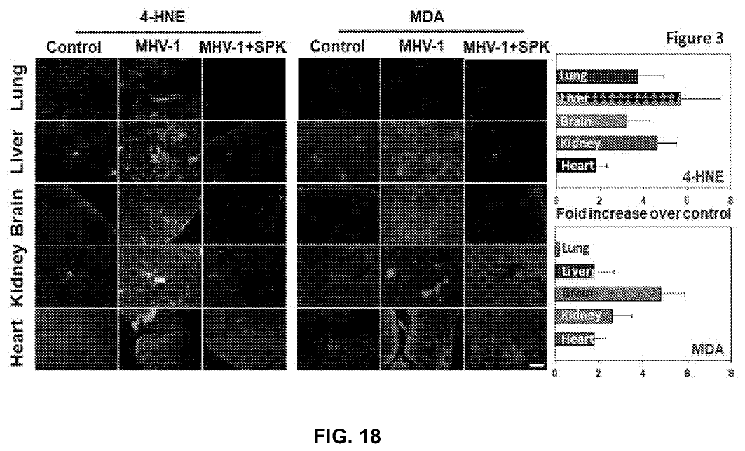

Lipid peroxidation-derived aldehydes, 4-hydroxynonenol (4-HNE)/malondialdehyde (MDA) were detected in control, MHV-1 inoculated, and MHV-1+SPK inoculated mice, as shown in FIG. 18. Treatment of MHV-1 infected mice with the SPK (5 mg/kg by wt; 3 injections from day 3 post-MHV-1, i.e., on days 3, 5, and 7) diminished lipid peroxidation (FIG. 18).

These findings suggested that oxidative stress could be a crucial factor involved in SARS-COV-2 infection, and that SPK, which was effective in preventing S1 binding with ACE2 or CCM, offers a potential preventive and therapeutic strategy for SARS-COV-2 infection.

### Example 7: Edema in the MHV-1 Mouse Model of SARS-COV-2 Infection

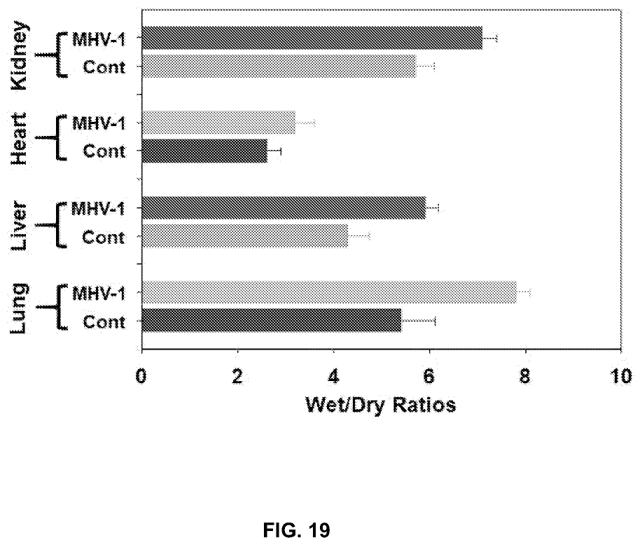

Protocol: Studies in humans with SARS-COV-2 infection have suggested a probable role of generalized edema in the pathogenesis of COVID-19. However, these studies were based on histopathological examination. The MHV-1 mouse model of SARS-COV-2 infection was utilized to investigate whether generalized edema also occurs and a mechanism by which MHV-1 induces edema. Results demonstrate increased edema of varying degrees in lung, brain, liver, kidney, and heart (FIG. 19). The edema was more severe in lung and brain, as compared to liver, kidney, and heart (FIG. 19). Further, treatment of MHV-1 infected mice with the SPK (5 mg/kg by wt; 3 injections from day 3 post-MHV-1, i.e., on days 3, 5 and 7) resulted in edema levels similar to controls.

These findings suggested that the development of edema in various organs may be a critical event in SARS-COV-2 infection, and that SPK, which was effective in preventing S1 binding with ACE2 or CCM, offers a potential preventive and therapeutic strategy for SARS-COV-2 infection.

### Example 8 Altered AQPs in Multiple Organs Post-MHV-1 Infection in Mice

Protocol: Expression of aquaporins (AQPs) was determined in mouse models as providing insight into the development of edema as intra- and extracellular water levels in disease conditions.

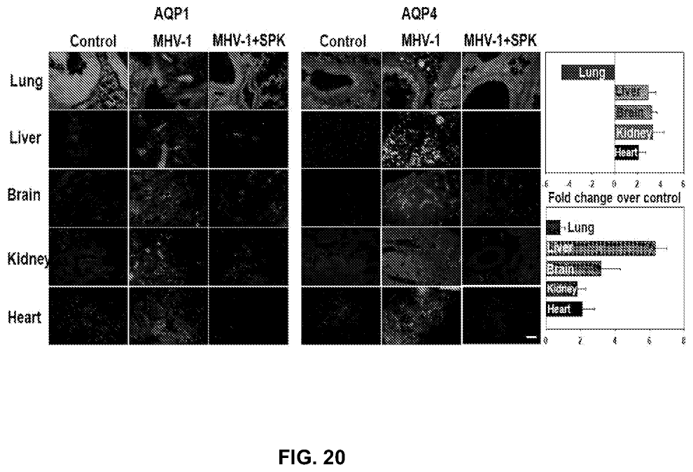

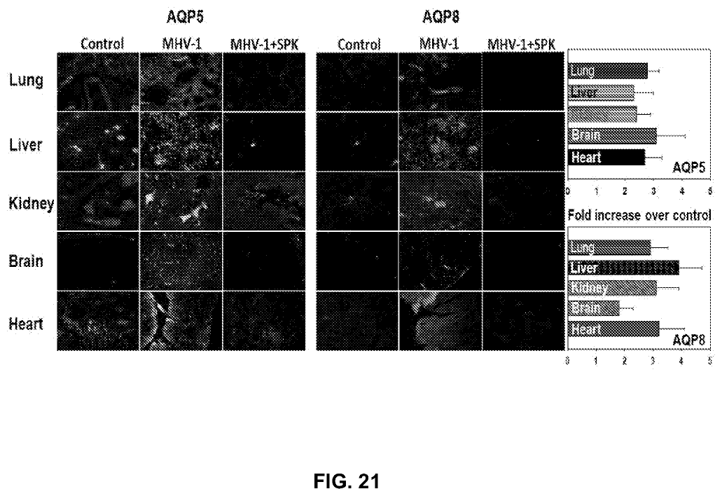

Results: AQPs 1, 4, 5 and 8 are increased in all organs examined (FIGS. 20 & 21) (Figure not shown for AQPs 1 and 4). Treatment with SPK reduced AQPs 1, 4, 5, and 8 levels.

The findings strongly suggest that altered AQPs in various organs is a crucial event in the pathogenesis of SARS-COV-2 infection and indicates the contributions of edema in the pathogenesis of COVID-19.

### Example 9. SPIKENET on SARS-COV-2 Infection

Protocol: K18-hACE2 transgenic mouse are examined to recapitulate events occurring in humans associated with SARS COV-2 infection, as well as to establish the MHV-1 in mice. Viral titer, various organ injury (histopathology), edema and AQP with and without SPK are measured. Animals are administered SPK one or more times per day. Additional markers can include body scoring, physiological function measurements, behavior analysis, and biological, chemical, and biochemical analysis. Biochemical testing can include tests indicative of organ function, including but not limited to skeletal, muscular, nervous, endocrine, cardiovascular, lymphatic, reparatory, digestive, urinary, and reproductive function. Disease and non-diseased animals are divided into control (non-treated); vehicle control (carrier without SPIKENET) and SPIKENET groups. Tissue histopathology, molecular biology, and protein chemistry analysis of tissues are also compared in control (non-treated); vehicle control (carrier without SPIKENET) and SPIKENET groups.

Improvements in organ injury (histopathology), edema and AQP with and without SPK are measured. In addition, improvements in body scoring, physiological function measurements, behavior analysis, and biological, chemical, and biochemical analysis will be demonstrated in animals receiving SPIKENET one or more times per day

### Example 10. Applicability of SPIKENET in Treatment of Other Conditions

Protocol: SPIKENET has demonstrated effectiveness against preventing SARS-COV-2 infection and against oxidative stress and multi-organ failure. The peptide is also neuroprotective, potent against preventing Lipopolysaccharide mediated events and effectiveness against edema mediated by changes in aquaporins. Using mouse models of diseases characterized by oxidative stress, multi-organ failures, LPS, and related diseases the effectiveness of SPIKENET was tested. Experiments similar to those for SARS-COV-2 are conducted and results scored for disease severity, fluid and tissue harvesting, physiological and functional monitoring, organ function tests, histopathology and biological, chemical, and biochemical analysis of animals and harvested organs and tissues. Biochemical tests indicative of organ function included, but not limited to skeletal, muscular, nervous, endocrine, cardiovascular, lymphatic, respiratory, digestive, urinary, and reproductive function. Disease and non-diseased animals will be divided into control (non-treated); vehicle control (carrier without SPIKENET) and SPIKENET groups. Animals received SPIKENET one or more times per day. Tissue histopathology, molecular biology, and protein chemistry analysis of tissues were compared in control (non-treated); vehicle control (carrier without SPIKENET) and SPIKENET groups.

Results Diseased animals demonstrate diminished physiological responses and disease scoring indicative of development of the disease. Histopathology, biochemical, and chemical analyses are characteristic of the disease being investigated. Animals treated with SPIKENET display measures similar to non-treated controls demonstrating reduced signs of disease, enhanced physiological function, and improved function of one or more of skeletal, muscular, nervous, endocrine, cardiovascular, lymphatic, respiratory, digestive, urinary, and reproductive function. Histopathological, protein chemistry, and molecular biology results demonstrate reductions in disease-specific markers; reduction in free radicals and overall improvements in inflammatory markers and indices of disease. Tissue edema is similar to control animals with disease scoring indicative of improvements in overall appearance and reductions in disease onset and severity. Improvements in cognitive and physiological functions and associated measures are present in animals receiving SPIKENET when compared to disease counterparts.

More specifically, SPIKENET eliminates/mitigates disease-related death and weight loss. SPIKENET mitigates one or more of lung inflammation, peribronchiolar interstitial infiltration, bronchiolar epithelial cell necrosis and intra-alveolar necrotic debris, alveolar exudation (surrounding alveolar walls have capillaries that are dilated and filled with RBCs), mononuclear cell infiltration, hyaline membrane formation, the presence of hemosiderin-laden macrophages, as well as interstitial edema. SPIKENET protection in the liver includes protection against one or more of severe liver vascular congestion, luminal thrombosis of portal and sinusoidal vessels, hepatocytes degeneration, cells necrosis and/or hemorrhagic changes. Kidney related benefits of SPIKENET include elimination/mitigation of one or more of proximal and distal tubular necrosis, hemorrhage in interstitial tissue, and vacuolation of renal tubules were observed in the kidney. SPIKENET administration in disease models is associated with elimination/mitigation of one or more of interstitial edema, vascular congestion and dilation, and red blood cells extravasation into interstitium. SPIKENET administration in disease models is associated with elimination/mitigation of congested blood vessels, perivascular cavitation, cortical pericellular halos, vacuolation of neuropils, darkly stained nuclei, pyknotic nuclei and associated vacuolation of the neuropil in the cortex, and acute eosinophilic necrosis and necrotic neurons with fragmented nuclei and vacuolation in the hippocampus.

Conclusion: These results demonstrate the efficacy of SPIKENET for use in a wide range of disease characterized by oxidative stress, multi-organ failure, neurodegeneration, inflammation, and coagulation.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention, as defined in the following claims.

1. A pharmaceutical formulation for treating or preventing a coronavirus infection in an animal or human comprising a therapeutically effective amount of a peptide, wherein the formulation comprises peptides having an amino acid sequence comprising:
(SEQ ID NO: 20)
(M)nVR(I/L)KP(G/A)(S/T)(A/G)NKP(S/T)(D/E)D,
wherein n=0 or 1, wherein the peptide does not have the amino acid sequence MVRIKPGSANKPSDD (SEQ ID NO: 21) and wherein variant amino acid sequence residues are set forth in the alternative at each substituted position.

2. A peptide having an amino acid sequence that is
(SEQ ID NO: 1)
MVRIKPASANKPSDD;
(SEQ ID NO: 2)
MVRIKPGTANKPSDD;
(SEQ ID NO: 3)
MVRIKPGTANKPTDD;
(SEQ ID NO: 4)
MVRIKPGSGNKPSDD;
(SEQ ID NO: 5)
MVRIKPATANKPSDD;
(SEQ ID NO: 6)
MVRIKPGTGNKPSDD;
(SEQ ID NO: 7)
MVRIKPGTGNKPTDD;
(SEQ ID NO: 8)
MVRIKPATANKPTDD;
(SEQ ID NO: 9)
MVRIKPATGNKPTDD;
(SEQ ID NO: 10)
MVRIKPATGNKPSDD;
(SEQ ID NO: 11)
VRLKPASANKPSED;
(SEQ ID NO: 12)
VRLKPASGNKPSED;
(SEQ ID NO: 13)
VRLKPGSGNKPSED;
(SEQ ID NO: 14)
VRLKPATGNKPTED;
(SEQ ID NO: 15)
VRLKPGTANKPTED;
(SEQ ID NO: 16)
VRLKPATANKPTED.
(SEQ ID NO: 17)
VRLKPGTGNKPTED;
(SEQ ID NO: 18)
VRLKPGTANKPSED;
or
(SEQ ID NO: 19)
VRIKPGTANKPSED
or salts or derivatives thereof.

3. The peptide of claim 1, having the amino acid sequence MVRIKPASANKPSDD (SEQ ID NO: 1) and salts and derivatives thereof.

4. A pharmaceutical formulation for treating or preventing a coronavirus infection in an animal or human comprising a therapeutically effective amount of a peptide of claim 1 and one or a plurality of excipients, adjuvants, or other formulation components.

5. A method of treating or preventing infection of a human or animal with a coronavirus, the method comprising administering to a human or animal in need thereof a pharmaceutical formulation of claim 4.

6. The method of claim 5, wherein the formulation is administered intranasally, intravenously, mucosally, orally subcutaneously or intramuscularly.

7. The method of claim 6, wherein the formulation is administered two or more times per day.

8. The method of claim 6 wherein the pharmaceutical formulation is administered to a human.

9. The method of claim 5, wherein the coronavirus is SARS-COV-2.

10. A pharmaceutical formulation for treating or preventing a coronavirus infection in an animal or human comprising a therapeutically effective amount of the peptide of claim 2 and one or a plurality of excipients, adjuvants, or other formulation components.

11. A method of treating or preventing infection of a human or animal with a coronavirus, the method comprising administering to a human or animal in need thereof the pharmaceutical formulation of claim 10.

12. The method of claim 11, wherein the pharmaceutical formulation is administered intranasally, intravenously, mucosally, orally subcutaneously or intramuscularly.

13. The method of claim 12, wherein the pharmaceutical formulation is administered two or more times per day.

14. The method of claim 12, wherein the pharmaceutical formulation is administered to a human.

15. The method of claim 11, wherein the coronavirus is SARS-COV-2.
